(19) United States
(12) Patent Application Publication
(10) Pub. No.: US 2023/0260614 A1
(43) Pub. Date: Aug. 17, 2023

(54) MOBILE APPLICATION TO PROVIDE REAL-TIME DATA OF TAPERING TREATMENT

(71) Applicant: OPOS, Inc., San Mateo, CA (US)

(72) Inventors: Justin KROMELOW, Kirkland, WA (US); Rao MIKKILINENI, Ph.D, Cupertino, CA (US); Julie CAREY, San Mateo, CA (US)

(21) Appl. No.: 18/159,618

(22) Filed: Jan. 25, 2023

Related U.S. Application Data
(60) Provisional application No. 63/303,942, filed on Jan. 27, 2022.

(51) Int. Cl. G16H 20/10; G16H 50/20; G16H 40/67; G06F 21/62; G06F 21/33

(52) U.S. Cl. CPC G16H 20/10; G06F 21/335; G06F 21/6254; G16H 40/67; G16H 50/20

(57) ABSTRACT

The present invention relates to a mobile application configured to collect data and monitor a patient in real-time, to identify tapering points in an opioid tapering treatment, comprising the steps of receiving at least one user detail at least in part with the medication, importing de-identified data associated with the patient, wherein the de-identified data is determined using a clinical decision support tool or one or more tapering algorithms, and generating and displaying a dashboard for review and probabilistic outcomes of customized taper plans for the patient.

### FIELD OF INVENTION

The present invention relates to a mobile application to collect data and monitor patients in real-time, and particularly detect and identify tapering points in an opioid tapering treatment.

### BACKGROUND

Opioids are widely used to manage chronic pain with almost 20 million people being prescribed a 30-day or longer prescription. According to the Center for Disease Control and Prevention (CDC), there are between 9 million and 18 million adults receiving opioids at a greater than recommended dosage. While higher dosages may be justified for some patients under the care of pain specialists, it does represent an increased mortality risk and increased risk of accidental overdose, wherein the CDC recommends using the lowest dose of opioids.

Conventionally, an opioid taper is opted to lower the opioid dosage and tapering is conducted in a medically supervised inpatient or outpatient setting. Existing methods are employed by observing the techniques used by pain specialists and combining that information with the patient experience using machine algorithms.

Thus, to provide a better, individualized and flexible taper plan based on individual patient requirements, there is a need for an effective and optimized method that can provide clinical decision support to primary care physicians with the context of the patient’s current circumstances and is further available at the ease of the personal computing devices to automatically notify a responder.

### OBJECTIVES OF THE INVENTION

The primary objective of the present invention is to provide a web-based application to monitor in real-time, within the course of the life cycle of tapering points by providing personalized treatment plans to reduce the functional impacts of medications used for chronic conditions.

Another objective of the present invention is to provide an application that includes a dashboard for providing alerts, monitoring status, and clinical decisions in support of recommendations and descriptions of probabilistic outcomes of customized taper plans.

Yet another objective of the present invention is to provide an application that functions as an opioid tapering decision support tool enabling primary care and pain specialists to review the patient’s status and accordingly assist the patient in minimizing opioid dosage.

### SUMMARY

Various embodiments of the present disclosure provide methods and systems for detecting and identifying tapering points in an opioid tapering treatment.

In an embodiment, a computer-implemented method is disclosed. The method performing the steps of storing in a nonvolatile memory of a mobile device an application program configured to manage opioid tapering treatment, accessing the nonvolatile memory to upload into a processor of the mobile device and run the application, and programming the processor with the uploaded application program for collecting data and receiving at least one patient detail at least in part with the medication, de-identifying the data by removing or modifying at least one element regarded as protected health information associated with the patient and adding a unique encrypted user token to each record, importing de-identified data associated with the patient, wherein the de-identified data is determined using a clinical decision support tool or one or more tapering algorithms, and generating and displaying a dashboard for review and probabilistic outcomes of customized taper plans for the patient.

In another embodiment, a system is disclosed. The system is configured to perform the steps of collecting and receiving data, by a processor, comprising at least one patient detail at least in part with the medication; de-identifying the data, by the processor, by removing or modifying at least one element regarded as protected health information associated with the patient; and adding a unique encrypted user token to each record; importing, by the processor, de-identified data associated with the patient, wherein the de-identified data is determined using a clinical decision support tool or one or more tapering algorithms; and generating and displaying, by the processor, a dashboard for review and probabilistic outcomes of customized taper plans for the patient.

In yet another embodiment, a non-transitory computer-readable medium whose contents cause at least one computer to perform a method for identifying tapering points in an opioid tapering treatment is disclosed. The method of collecting and receiving data, by a processor, comprising at least one patient detail at least in part with the medication; de-identifying the data, by the processor, by removing or modifying at least one element regarded as protected health information associated with the patient; and adding a unique encrypted user token to each record; importing, by the processor, de-identified data associated with the patient, wherein the de-identified data is determined using a clinical decision support tool or one or more tapering algorithms; and generating and displaying, by the processor, a dashboard for review and probabilistic outcomes of customized taper plans for the patient.

### BRIEF DESCRIPTION OF THE DRAWINGS

The disclosed embodiments have other advantages and features which will be more readily apparent from the following detailed description of the invention and the appended claims, when taken in conjunction with the accompanying drawings, in which:

FIG. 1 is a diagram of the environment of an application for an opioid taper treatment matching platform, according to one embodiment;

FIG. 2 is a block diagram showing the functional components of an application for an opioid taper treatment matching platform, according to one embodiment;

FIG. 3 is a flowchart illustrating an example method, according to one embodiment; and

FIG. 4 is a block diagram illustrating components of an example machine able to read instructions from a machine-readable medium and execute them in a processor (or controller).

### DETAILED DESCRIPTION

The figures and the following description relate to preferred embodiments by way of illustration only. It should be noted from the following discussion, that alternative embodiments of the structures and methods disclosed herein will be readily recognized as viable alternatives and may be employed without departing from the principles of what is claimed.

In the present disclosure, the word ‘subject’ refers to a patient, who is being diagnosed for detection of opioid dosage and or is undergoing an opioid taper treatment.

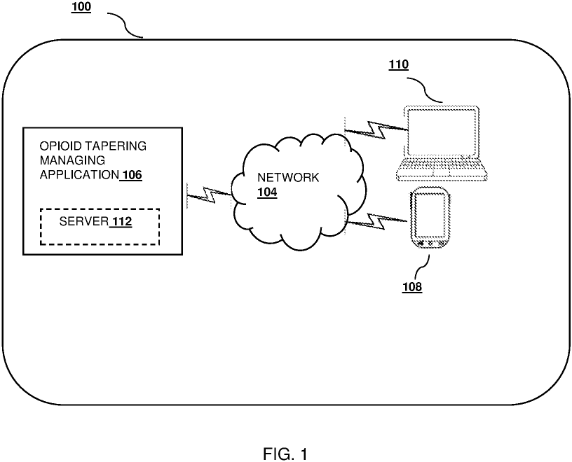

FIG. 1 illustrates an example environment of a mobile application configured to provide real-time data of tapering treatment, according to one example embodiment. The environment 100 may include a network 104, an application 106, and one or more user devices 108 and 110. The user devices 108 and 110, are communicatively connected through a network 104 via the application 106.

Network 104 may include, for example, one or more of the Internet, Wide Area Networks (WANs), Local Area Networks (LANs), analog or digital wired and wireless telephone networks (e.g., a public switched telephone network (PSTN), Integrated Services Digital Network (ISDN), a cellular network, and Digital Subscriber Line (DSL)), radio, television, cable, satellite, or any other delivery or tunneling mechanism for carrying data. Network 104 may include multiple networks or subnetworks, each of which may include, for example, a wired or wireless data pathway. Network 104 may include a circuit-switched network, a packet-switched data network, or any other network able to carry electronic communications (e.g., data or voice communications.) For example, network 104 may include networks based on the Internet protocol (IP), asynchronous transfer mode (ATM), the PSTN, packet-switched networks based on IP, X.25, or Frame Relay, or other comparable technologies and may support voice using, for example, VoIP, or other comparable protocols used for voice communications. Network 104 may include one or more networks that include wireless data channels and wireless voice channels. Network 104 may be a wireless network, a broadband network, or a combination of networks including a wireless network and a broadband network.

Various entities in the environment 100 may connect to the network 104 in accordance with various wired and wireless communication protocols, such as Transmission Control Protocol and Internet Protocol (TCP/IP), User Datagram Protocol (UDP), 2nd Generation (2G), 3rd Generation (3G), 4th Generation (4G), 5th Generation (5G) communication protocols, Long Term Evolution (LTE) communication protocols, or any combination thereof.

Application 106 may be, for example, a web interface or a standalone software application. Application 106 is coupled to server 112, wherein server 112 includes a processor and memory. Server 112 is configured to perform one or more of the operations described herein.

In one embodiment, the server system 112 is configured to enable the collection of data and to monitor patients or subjects in real-time, to identify tapering points in an opioid tapering treatment. Server system 112 is configured to collect de-identified data imported using a clinical decision support tool and/or implementing one or more tapering algorithms.

Server system 112 is configured to monitor a subject for opioid tapering management and may comprise one or more computing devices associated with an opioid tapering management service. The opioid tapering monitoring service can be configured to identify opioid monitoring information to identify tapering points and from at least one physiological or psychological parameter associated with the subject, where the opioid monitoring information comprises one of the tapering/weaning side effects, psychological or behavioral supports, tapering abandonment, an overdose alert, and a non-distress status. The status may be notified to at least a contact information associated with the overdose alert and the non-distress status. According to an embodiment, notifications are delivered to the subject, to a crowd-sourced community of friends, family, and other opioid users that have also downloaded the application onto their computing devices, and to emergency providers and medical care personnel.

According to an embodiment, an individual user may register via an initial sign-up. During registration, the user receives a welcome package including devices, set-up instructions, and information, as well as the capability to download a mobile application 106 or other integration data systems. Following the registration, the server system collects information about baseline behaviors, dosage details, tapering triggers and disease markers from different sources, and de-identifies this data by removing or modifying all elements regarded as protected health information, and adds a unique encrypted patient token to each record.

Using data in the de-identified data, the server system 112 generates an initial treatment plan by merging the matching unique encrypted user tokens of opioid health care data sets with the unique encrypted user tokens of the previously de-identified healthcare data sets. In an exemplary scenario, data analytics and algorithms are implemented to facilitate a personalized initial treatment and monitor and integrate a user’s compliance and response to the treatment plan.

The integration allows for treatment plan iteration and advancement using higher-level algorithms and analytical support customized by the data pulled into the system and based on user behaviors. When the user reaches a remission or cure phase, the system reaches a step of maintaining the response. Clinical definitions for remission and length of time specified for cure are treatment and subject dependent. Thus, the system can continue to monitor markers of disease and reinitiate treatment if a user’s lifestyle changes or if the triggers worsen.

The server system 112 via the application 106 can also alert the user and/or person(s) designated by the user to an abnormal data reading or a trigger. For example, an abnormally low blood oxygen saturation reading can cause the mobile computing device to buzz, vibrate or otherwise notify the user of an abnormal reading, and to transmit a notification or alert to the user, the designated person(s) or medical personnel to a network via the network connectivity 104.

The server system 112 is further configured as an opioid tapering decision support tool enabling primary care specialists to review the patient’s status through a constant feedback loop and accordingly assist the patient or subject in minimizing opioid dosage. The server system 112 may include algorithms to conduct randomized clinical studies to determine the safety and effectiveness of the tapering tool.

According to an embodiment, the server system 112 learns about the medical resources available, based on geographic or technological availability, employing one or more machine learning algorithms and lists to include only those resources that are available.

The server system 112 is configured to generate a report and display it as a dashboard for providing alerts, monitoring status, clinical decision support recommendations, and descriptions of probabilistic outcomes of customized taper plans, which can be accessed by the user (e.g., primary specialists or patient) for periodically monitoring and tracking the treatment process. The dashboard may include a graph indicating physiological or psychological parameters (e.g. oxygen saturation levels) and one or more of a phone number of the user, a location of the user, directions to the location of the user, the closest location of naloxone or other medication used to reverse the effects of an opioid overdose. The dashboard can also include an alert option that generates a notification as an automatic call to emergency responders.

The dashboard details may be utilized by medical professionals. Also, the reports may be transmitted to communication devices such as but not limiting to mobile phones and the like, for the user.

The user devices 108 and 110 may include hardware, software, or a combination thereof operated by one or more users and provides functionality for communicating with the application 106. The user devices 108 and 110 may be configured to install an application 106. The application may comprise of an algorithm, software, computer code, and/or the like, for example, mobile application software.

Further, the users of application 106 may include, but are not limited to, practitioners (e.g. physicians), practitioner support staff (e.g. nursing assistant, patient care attendant, medical assistant, aide, medical secretary, home care aide, or other staff), and other users (e.g. patients). Accordingly, electronic devices operated by the practitioner, practitioner support staff, patient, and other users may be in communication with the platform.

A user such as a practitioner, practitioner support staff, patient, or other users, may access server 112 through the application 106. The application 106 may be embodied as, for example, but not be limited to, a website, a web application, a desktop application, and a mobile application compatible with the user devices 108 & 110.

In other embodiments, server 112 may be incorporated, in whole or in part, into one or more parts of environment 100. In addition, the server system 112 should be understood to be embodied in at least one computing device in communication with the network 104, which may be specifically configured, via executable instructions, to perform as described herein, and/or embodied in at least one non-transitory computer-readable media.

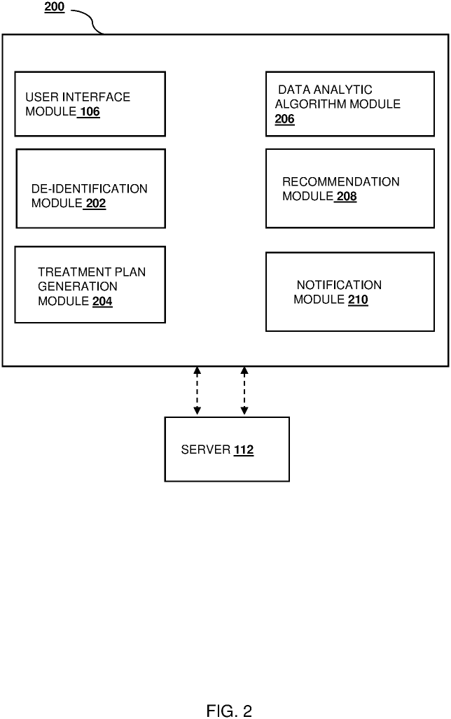

FIG. 2 is a block diagram illustrating various functional components of a server system 200 is shown, in accordance with an embodiment of the present disclosure. Server system 200 is similar to server system 112. In some embodiments, the server system 200 is embodied as a cloud-based and/or SaaS-based (software as a service) architecture.

It is noted that the server system 200 as illustrated and hereinafter described is merely illustrative of an apparatus that could benefit from embodiments of the present disclosure and, therefore, should not be taken to limit the scope of the present disclosure. It is noted that the server system 200 may include fewer or more components than those depicted in FIG. 2.

According to an embodiment, the server system 200 can include a de-identification module 202, a treatment plan generation module 204, a data analytic algorithm module 206, a recommendation module 208, a notification module 210, and a feedback module 212.

The de-identification module 202 is configured to collect and gather subject’s data from different sources and de-identify this data by removing or modifying all elements regarded as protected health information and adds a unique encrypted subject token to each record. After data records from other data sources are merged with the health data, all the PHI (Protected Health Information) and PII (Personally-Identifying Information) are removed from the merged health data and a unique token that may be encrypted is associated with the merged data.

In an embodiment, the tokenized data is merged with other healthcare data sets that have similarly been de-identified and tokenized by matching the unique encrypted person tokens in each data set with one another.

The treatment plan generation module 204 is configured to identify taper points and generate opioid tapering treatment plans for patients or similar drugs such as benzodiazepine. The treatment generation module 204 can include computer-executable instructions that, when executed, cause one or more processors to generate the treatment plan for the patient. For example, the treatment generation module 204 can be a programming objective that includes data and computer-executable instructions.

The treatment generation module 204 generates the treatment plan by assessing previously imported de-identified data, wherein the data may be imported using any clinical decision support tool and/or one or more tapering algorithms. The parameters for plans are generated by the treatment plan generation module 204 responsive to the observation of the patient profile, and current stage of the patient along with the medication dosage and/or rate adjustments, management of tapering/weaning side effects, psychological or behavioral support, tapering abandonment and/or patient profile type. The treatment generation module stores the merged details in a database until each of the data is transmitted to the data analytic algorithm module.

The data analytic algorithm module 206 is configured to calculate the analytical data for the received de-identified data. The data analytic algorithm module 206 calculates the treatment plan success rate based on the de-identified data associated with the subject. For example, the data analytic algorithm module 206, based on the past subject health information that is similar to the subject health information, calculates success rates for possible treatment plans.

The data analytic algorithm module 206 calculates the overall success rate of the treatment plans and calculates a clinician’s success rate for a treatment plan. In some examples, data analytic algorithm module 206 calculates a clinician success rate for all clinicians available to administer the treatment to the patient. The clinician’s success rate can be plotted. According to an embodiment, the success rate information may be graphically presented.

The recommendation module 208 is configured to refine the treatment plans, initiatives, and other suggested courses of action based on the analysis of data sources. Recommendation module 208 updates and lists the effective and relevant treatment plans correlated to the de-identified data made available by data sources

The notification module 210 enables a user to notify friends, family, and caregivers when they are in need of emergency care due to indications that an opioid overdose is imminent or occurring. For example, treatment centers, caregivers, and ambulance services can receive possible overdose alerts in order to provide immediate life-saving care to the user, an on-site caregiver can receive care instructions, friends and family can receive periodic status messages indicating no overdose event occurring, and transportation services can receive messages with the location of medications used to reverse the effects of an opioid overdose, such as naloxone, buprenorphine, a combination of buprenorphine and naloxone, and the like.

The feedback module 212 is configured to run feedback loops consistently to collect medical, psycho-social, behavioral, physiological and demographic. The feedback module 212 measures the compliance of the intended behavior with the treatment plan. Examples of behavioral monitoring for compliance include sleep and physical activity monitors and self-reported nutritional intake. The feedback module 212 assesses whether the algorithms and methodology correctly matched the recommendations to a person’s ability, motivation, preferences, and baseline lifestyle. The feedback module 212 frequently measures the treatment response, and captures real-time data on the effectiveness of the treatment plan(s) as based on the health improvement and can iterate on them, as desired, to achieve better results. The feedback loops provide training sets for future machine learning to refine the treatment plan and algorithms associated therewith. Both data on compliance and disease response are made available to the end-user through the web and mobile user interfaces.

The aforementioned modules may be used and interconnected in various combinations with one another. By way of a non-limiting example, any of the application modules may be interconnected and/or used in conjunction with at least one of the user interface modules, a patient tracking module, external data, or datasets. The application modules comprise at least one of the following modules, a recommendation module, a patient tracking module, a predictive analytics module, and a user interface module. The modules may be in communication with a central server In an embodiment, the modules may reside within a computer-readable medium, a server, a computer, a phone, a mobile device, a wearable device, or other electronic devices

In another embodiment, the modules can also be used together in conjunction and/or interconnected as separate elements of a system. In another embodiment, the modules may be interconnected and/or configured and/or working together as separate elements or components, while not part of or contained within the same component. In other embodiments, the modules may be contained within the same component and/or interconnected within one component or portion of a component and/or components.

In an embodiment, the module may be configured and/or used as an element and/or elements of a system. In yet other embodiments, functionality for any of the aforementioned modules can be interconnected such that the functionality is embodied in one individual module alone or collectively in a portion and or a subset of the modules. The modules can be embodied as software, hardware or a combination thereof. The modules can also be collectively embodied in one device, server, computer-readable medium, or hardware element.

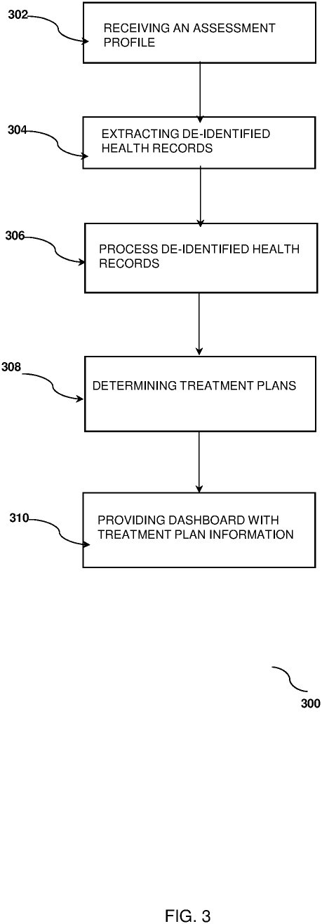

FIG. 3 is a flow diagram 300 representing the method to identify tapering points in an opioid tapering treatment according to an embodiment. The server system 112/200 is configured to perform a method, using a processor, that includes receiving 302 an assessment profile for analyzing opioid risks for a subject and extracting 304 de-identified health records based on the assessment profile. The method also includes processing 306 the de-identified health records to determine a set of goals, a set of outcomes, and a set of risk factors based on the subject’s health and behavior. The method additionally includes determining 308, based on the set of goals, the set of outcomes, and the set of risk factors, a plurality of recommended treatment plans. The method further includes providing 310 a user interface comprising a dashboard with information associated with the treatment plan set of goals and the set of outcomes, information associated with the set of risk factors, information with the plurality of tasks, and acting as a treatment tracking tool.

To provide the treatment tracking tool, server 112 is configured to extract and determine a first listing of past treatment approaches for chronic pain, and a second listing of current treatment approaches for chronic pain, analyze the first listing and the second listing to determine a third listing of future treatment approaches, and provide the treatment tracking tool within the listing of identified risks.

If the user demonstrates an improvement in individual condition(s) regardless of compliance, the server system 112 may run an analysis on the user’s behaviors to understand which behaviors are most correlated with health improvement. The results of this regression may be used to iterate the user’s treatment plan and/or system’s algorithms and may be presented to the end-user for positive reinforcement of healthy behaviors. If the user does not show an improvement but appears to be compliant with the treatment plan, the treatment plan may be advanced to a more aggressive intervention. If the user does not show an improvement and also is not compliant with the treatment plan, server system 112 may go into troubleshooting mode. The user may then be presented with reasons for not completing the intended behavior (e.g. preference for a different intervention, not enough time, lack of understanding, etc.) and the treatment plan is adjusted based on the reported reason. If the user does not engage with the troubleshooting mode, customer service representatives may reach out to better understand the situation.

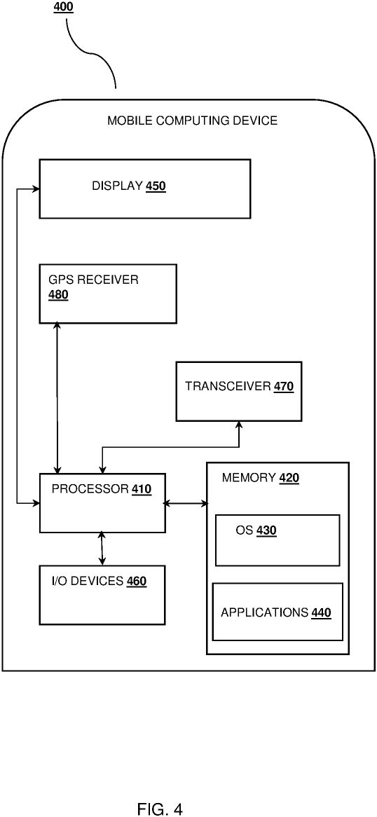

FIG. 4 is a block diagram illustrating the mobile computing device 400, according to an example embodiment. The device may correspond to, for example, one or more client machines or application servers. The mobile computing device 400 may include a processor 410. The processor 410 may be any of a variety of different types of commercially available processors suitable for mobile devices (for example, an XScale architecture microprocessor, a Microprocessor without Interlocked Pipeline Stages (MIPS) architecture processor, or another type of processor). A memory 420, such as a Random Access Memory (RAM), a Flash memory, or other types of memory, is typically accessible to the processor 410. The memory 420 may be adapted to store an operating system (OS) 430, as well as application programs 440, such as a mobile location-enabled application that may provide location-based services to a user.

The processor 410 may be coupled, either directly or via appropriate intermediary hardware, to a display 450 and to one or more input/output (I/O) devices 460, such as a keypad, a touch panel sensor, a microphone, and the like. Similarly, in some embodiments, the processor 410 may be coupled to a transceiver 470 that interfaces with an antenna 490. The transceiver 470 may be configured to both transmit and receive cellular network signals, wireless data signals, or other types of signals via the antenna 490, depending on the nature of the mobile device 400. Further, in some configurations, a GPS receiver 480 may also make use of the antenna 490 to receive GPS signals.

Upon reading this disclosure, those of skill in the art will appreciate additional alternative structural and functional designs for a system and a process for automated identification and selection mechanism for candidates in a recruitment process through the disclosed principles herein. Thus, while particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the method and apparatus disclosed herein without departing from the spirit and scope defined in the appended claims.

What is claimed:

1. A method for identifying tapering points in an opioid tapering treatment, using a mobile application, comprising the steps of:
storing in a nonvolatile memory of a mobile device an application program configured to manage opioid tapering treatment;
accessing the nonvolatile memory to upload into a processor of the mobile device and run the application; and
programming the processor with the uploaded application program for:
collecting data and receiving at least one patient detail at least in part with the medication;
de-identifying the data by removing or modifying at least one element regarded as protected health information associated with the patient and adding a unique encrypted user token to each record;
importing de-identified data associated with the patient, wherein the de-identified data is determined using a clinical decision support tool or one or more tapering algorithms; and
generating and displaying a dashboard for review and probabilistic outcomes of customized taper plans for the patient.

2. The method for identifying tapering points in an opioid tapering treatment of claim 1, further comprising, monitoring the patient for opioid tapering management, using one or more computing devices associated with an opioid tapering management service.

3. The method for identifying tapering points in an opioid tapering treatment of claim 1, wherein identifying tapering points includes at least one physiological or psychological parameters associated with the patient.

4. The method for identifying tapering points in an opioid tapering treatment of claim 1, wherein the opioid monitoring information comprises at least one of:
the tapering or weaning side effects;
psychological or behavioral supports;
tapering abandonment; and
an overdose alert or a non-distress status.

5. The method for identifying tapering points in an opioid tapering treatment of claim 1, further comprises: generating and delivering notifications to at least one of the patients, a crowd-sourced community of members, emergency providers, and medical care personnel.

6. The method for identifying tapering points in an opioid tapering treatment of claim 1, wherein the data collected includes information about baseline behaviors, dosage details, tapering triggers and disease markers.

7. The method for identifying tapering points in an opioid tapering treatment of claim 1, further comprising, generating an initial treatment plan by merging the matching unique encrypted user tokens of opioid health care data sets with the unique encrypted user tokens of the previously de-identified healthcare data sets.

8. The method for identifying tapering points in an opioid tapering treatment of claim 1, further comprising, iterating the treatment plans using higher-level algorithms and analytical support customized from the collected data and patient’s behavior.

9. The method for identifying tapering points in an opioid tapering treatment of claim 1, further comprises, enabling primary care specialists to review the patient’s status through a constant feedback loop and accordingly assist the patient in minimizing opioid dosage.

10. A system for identifying tapering points in an opioid tapering treatment for a patient comprising:
collecting data and receiving, by a processor, at least one patient detail at least in part with the medication;
de-identifying, by the processor, the data by removing or modifying at least one element regarded as protected health information associated with the patient and adding a unique encrypted user token to each record;
importing, by the processor, de-identified data associated with the patient, wherein the de-identified data is determined using a clinical decision support tool or one or more tapering algorithms; and
generating and displaying, by the processor, a dashboard for review and probabilistic outcomes of customized taper plans for the patient.

11. The system for identifying tapering points in an opioid tapering treatment of claim 10, further comprising, monitoring, by the processor, the patient for opioid tapering management using one or more computing devices associated with an opioid tapering management service.

12. The system for identifying tapering points in an opioid tapering treatment of claim 10, wherein identifying tapering points includes at least one physiological or psychological parameter associated with the patient.

13. The system for identifying tapering points in an opioid tapering treatment of claim 10, where the opioid monitoring information comprises at least one of:
the tapering or weaning side effects;
psychological or behavioral supports;
tapering abandonment; and
an overdose alert or a non-distress status.

14. The system for identifying tapering points in an opioid tapering treatment of claim 10, further comprising: generating and delivering, by the processor, notifications to at least one of: the patient, a crowd-sourced community of members, emergency providers and medical care personnel.

15. The system for identifying tapering points in an opioid tapering treatment of claim 10, wherein the data collected includes information about baseline behaviors, dosage details, tapering triggers, and disease markers.

16. The system for identifying tapering points in an opioid tapering treatment of claim 10, further comprises, generating, by the processor, an initial treatment plan by merging the matching unique encrypted user tokens of opioid health care data sets with the unique encrypted user tokens of the previously de-identified healthcare data sets.

17. The system for identifying tapering points in an opioid tapering treatment of claim 10, further comprising iterating, by the processor, of the treatment plans, using higher-level algorithms and analytical support customized from the collected data and patient’s behavior.

18. The method for identifying tapering points in an opioid tapering treatment of claim 10, further comprises, enabling, the processor, and primary care specialists to review the patient’s status through a constant feedback loop and accordingly assist the patient in minimizing opioid dosage.

19. A non-transitory computer-readable medium whose contents cause at least one computer to perform a method for identifying tapering points in an opioid tapering treatment, the method comprising:
collecting data and receiving, by a processor, at least one patient detail at least in part with the medication;
de-identifying, by the processor, the data by removing or modifying at least one element regarded as protected health information associated with the patient and adding a unique encrypted user token to each record;
importing, by the processor, de-identified data associated with the patient, wherein the de-identified data is determined using a clinical decision support tool or one or more tapering algorithms; and
generating and displaying, by the processor, a dashboard for review and probabilistic outcomes of customized taper plans for the patient.

20. The method for identifying tapering points in an opioid tapering treatment of claim 19, further comprising, generating, by the processor, an initial treatment plan by merging the matching unique encrypted user tokens of opioid health care data sets with the unique encrypted user tokens of the previously de-identified healthcare data sets.
